(19) United States
(12) Patent Application Publication
(10) Pub. No.: US 2023/0063970 A1
(43) Pub. Date: Mar. 2, 2023

(54) IRON-CONTAINING COMPOSITION

(71) Applicants: Yasutaka SUGIYAMA, Yokkaichi-shi, Mie (JP); Shiori EMA, Yokkaichi-shi, Mie (JP); Tomohisa FUKUHARA, Mie-gun, Mie (JP); Takeshi NAKAMURA, Mie-gun, Mie (JP)

(72) Inventors: Yasutaka SUGIYAMA, Yokkaichi-shi, Mie (JP); Shiori EMA, Yokkaichi-shi, Mie (JP); Tomohisa FUKUHARA, Mie-gun, Mie (JP); Takeshi NAKAMURA, Mie-gun, Mie (JP)

(73) Assignee: TAIYO KAGAKU CO., LTD., Yokkaichi-shi, Mie (JP)

(21) Appl. No.: 17/789,595

(22) PCT Filed: Jan. 8, 2021
(86) PCT No.: PCT/JP2021/000538

§ 371 Date: Jun. 28, 2022

(30) Foreign Application Priority Data
Jan. 31, 2020 (JP) 2020-015138

(51) Int. Cl. A23L 29/00; A23L 29/25

(52) U.S. Cl. CPC A23L 29/015; A23L 29/25; A23L 29/035

(57) ABSTRACT

An iron-containing composition containing ferric pyrophosphate, a polyhydric alcohol, gum arabic, and gum ghatti, wherein the content of the polyhydric alcohol is from 93 to 630 parts by mass, based on 100 parts by mass of the ferric pyrophosphate, and wherein a total amount of gum arabic and gum ghatti is from 15 to 23 parts by mass, based on 100 parts by mass of the ferric pyrophosphate, and wherein a mass ratio of the content of the gum arabic to the content of the gum ghatti (gum arabic/gum ghatti) is from 0.01 to 0.5, and wherein the ferric pyrophosphate has a volume-average particle size of 0.20 μm or less. The iron-containing composition of the present invention can be used in foodstuff, feeds, cosmetics, pharmaceutical compositions, and the like.

### TECHNICAL FIELD

The present invention relates to an iron-containing composition and a method for producing the same, and foodstuff containing an iron-containing composition.

### BACKGROUND ART

Conventionally, various compositions for fortifying iron in foodstuff, feeds, cosmetics, pharmaceutical compositions, or the like have been proposed.

For example, Patent Publication 1 discloses a food additive slurry composition containing ferric pyrophosphate and a particular amount of gum arabic and satisfying the requirements of a specified calcium ion concentration, as a food additive slurry composition having high concentration and high dispersibility. In addition, Patent Publication 2 discloses a composition for iron-fortified foodstuff characterized in that only gum ghatti in a particular amount based on a water-insoluble iron salt is used, as a composition for iron-fortified foodstuff that maintains excellent taste of the foodstuff themselves without showing a metallic taste of iron in the foodstuff, and has excellent dispersibility and effectively inhibits secondary aggregation during storage.

### PRIOR ART REFERENCES

### Patent Publications

Patent Publication 1: Japanese Patent Gazette No. 3512113
Patent Publication 2: Japanese Patent Gazette No. 4833176

### SUMMARY OF THE INVENTION

### Problems to be Solved by the Invention

However, in the compositions disclosed in Patent Publications 1 and 2, it has been found out that if the particle sizes of the iron agent are reduced to 0.2 μm or so in order to further make dispersibility favorable, the viscosity becomes undesirably high, so that there are some disadvantages in productivity such as the production at high concentrations cannot be performed. Therefore, further improvements on the dispersibility and the productivity in iron-containing compositions are desired.

The present invention relates to the provision of an iron-containing composition having excellent dispersibility and productivity.

### Means to Solve the Problems

The present invention relates to an iron-containing composition containing ferric pyrophosphate, a polyhydric alcohol, gum arabic, and gum ghatti, wherein the content of the polyhydric alcohol is from 93 to 630 parts by mass, based on 100 parts by mass of the ferric pyrophosphate, and wherein a total amount of the gum arabic and the gum ghatti is from 15 to 23 parts by mass, based on 100 parts by mass of the ferric pyrophosphate, and wherein a mass ratio of the content of the gum arabic to the content of the gum ghatti is from 0.01 to 0.5, and wherein the ferric pyrophosphate has a volume-average particle size of 0.20 μm or less.

### Effects of the Invention

According to the present invention, an iron-containing composition having excellent dispersibility and productivity can be provided.

### MODES FOR CARRYING OUT THE INVENTION

As a result of studying the above problems, the present inventors have newly found that even when the particle sizes of the iron agent are reduced, the composition can be used at high concentrations by using ferric pyrophosphate as an iron agent and using specified amounts of a polyhydric alcohol, gum arabic, and gum ghatti, whereby both dispersibility and productivity can be satisfied.

The iron-containing composition of the present invention contains ferric pyrophosphate, a polyhydric alcohol, gum arabic, and gum ghatti.

The content of the ferric pyrophosphate in the iron-containing composition of the present invention can be properly set depending upon the applications. For example, during the production of the iron-containing composition, it is preferable that the composition is prepared at a high concentration of the ferric pyrophosphate, such as preferably from 15 to 25% by mass, and more preferably from 20 to 25% by mass, from the viewpoint of productivity. In addition, in a case of a composition for foodstuff, the ferric pyrophosphate can be used at a concentration of from 1 to 20% by mass, preferably from 5 to 15% by mass, or the like, and the composition may be prepared at the above concentration, or a composition can also be prepared by diluting an iron-containing composition prepared at high concentrations. The state of the iron-containing composition of the present invention may be a liquid or a powder. In the embodiment of a liquid state, water can be contained, and in the embodiment of a powder state, an excipient such as dextrin can also be added. The content of the ferric pyrophosphate as used herein is measured in accordance with a method for quantifying ferric pyrophosphate described in JAPAN'S SPECIFICATION AND STANDARDS FOR FOOD ADDITIVES. Here, the iron-containing composition of the present invention may contain an iron agent other than the ferric pyrophosphate within the range that would not impair the effects of the present invention, and the iron agent preferably essentially only consists of ferric pyrophosphate. The other iron agents include ferric phosphate, ferrous phosphate, ferrous pyrophosphate, ferric hydroxide, ferric oxide, and the like.

The ferric pyrophosphate in the iron-containing composition of the present invention is formed into microparticles by a known pulverizing means such as a wet pulverizer such as a beads-mill, a sand-mill, or Coball Mill, an emulsifier-disperser such as Nanomizer, STAR BURST, Microfluidizer, or Homogenizer, or a ultrasonic disperser. The volume-average particle size of the ferric pyrophosphate in the iron-containing composition of the present invention is 0.20 μm or less, preferably 0.18 μm or less, and more preferably 0.16 μm or less, from the viewpoint of dispersibility, and the volume-average particle size is preferably 0.10 μm or more, more preferably 0.12 μm or more, and even more preferably 0.14 μm or more, from the viewpoint of productivity, and the volume-average particle size may be within the range of any of the combinations of the above. The volume-average particle size of the ferric pyrophosphate as used herein is measured with a laser diffraction type particle size distribution measurement apparatus LS13 320, manufactured by BECKMAN COULTER.

The polyhydric alcohol contained in the iron-containing composition of the present invention includes compounds having 2 to 12 carbon atoms and having two or more hydroxyl groups in one molecule, and sugar alcohols. The polyhydric alcohol includes, for example, ethylene glycol, propylene glycol, dipropylene glycol, glycerol, diglycerol, triglycerol, polyglycerol, ethylene glycol, diethylene glycol, polyethylene glycol, 3-methyl-1,3-butanediol, 1,3-butylene glycol, sorbitol, xylitol, maltitol, lactitol, sorbitan, xylose, arabinose, mannose, lactose, sugar, glucose, enzymatically hydrolyzed glucose syrup, acid-hydrolyzed saccharified glucose syrup, maltose starch syrup, maltose, high-fructose corn syrup, fructose, reduced maltose starch syrup, reduced starch glucose starch syrup, honey, fructose glucose syrup, and the like. Among them, propylene glycol, glycerol, diglycerol, triglycerol, and polyglycerol are preferred, from the viewpoint of easiness in the preparation of the formulation and stability, and propylene glycol and glycerol are more preferred, from the viewpoint that the polyhydric alcohol can be used in foods.

The content of the polyhydric alcohol in the iron-containing composition of the present invention can be properly set within the range of from 93 to 630 parts by mass, and preferably from 100 to 630 parts by mass, based on 100 parts by mass of the ferric pyrophosphate. For example, when the content of the ferric pyrophosphate in the iron-containing composition of the present invention is at a high concentration such as from 15 to 25% by mass, the polyhydric alcohol preferably has a low content such as from 93 to 330 parts by mass, or from 100 to 330 parts by mass, and it is preferable that the polyhydric alcohol is contained in a larger amount as the ferric pyrophosphate has a low concentration. For example, when the content of the ferric pyrophosphate is from 10 to 15% by mass, and preferably from 12 to 13% by mass, the content of the polyhydric alcohol is preferably from 300 to 460 parts by mass, more preferably from 330 to 460 parts by mass, and even more preferably from 360 to 460 parts by mass. In addition, when the content of the ferric pyrophosphate is from 5 to 10% by mass, and preferably from 7 to 9% by mass, the content of the polyhydric alcohol is preferably from 500 to 630 parts by mass. The content when two or more kinds of the polyhydric alcohols are contained is a total amount of the polyhydric alcohols.

A total amount of the gum arabic and the gum ghatti in the iron-containing composition of the present invention, based on 100 parts by mass of the ferric pyrophosphate, is 15 parts by mass or more, preferably 16 parts by mass or more, and more preferably 18 parts by mass or more, from the viewpoint of dispersibility, and the total amount is 23 parts by mass or less, and preferably 22 parts by mass or less, from the same viewpoint, and the total amount may be within any of the combinations of the above.

The mass ratio of the content of the gum arabic to the content of the gum ghatti (gum arabic/gum ghatti) in the iron-containing composition of the present invention is 0.01 or more, and preferably 0.02 or more, from the viewpoint of dispersibility, and the mass ratio is 0.50 or less, preferably 0.40 or less, more preferably 0.30 or less, and even more preferably 0.15 or less, from the same viewpoint, and the mass ratio may be within the range of any of the combinations of the above.

The contents of the gum arabic and the gum ghatti in the iron-containing composition of the present invention may be those satisfying the total amount and the ratio mentioned above, and the content of the gum arabic is preferably from 0.3 to 5.0 parts by mass, more preferably from 0.3 to 1.0 part by mass, and even more preferably from 0.3 to 0.5 parts by mass, based on 100 parts by mass of the ferric pyrophosphate, and the content of the gum ghatti is preferably from 15 to 23 parts by mass, and more preferably from 16 to 22 parts by mass, based on 100 parts by mass of the ferric pyrophosphate.

The iron-containing composition of the present invention can optionally contain a thickening polysaccharide other than the gum arabic and the gum ghatti. The optional thickening polysaccharides as mentioned above include, but not particularly limited to, xanthan gum, guar gum, tamarind gum, locust bean gum, carrageenan, pectin, glucomannan, alginic acid, curdlan, karaya gum, psyllium seed gum, gellan gum, Tara gum, pullulan, and decomposition products thereof, and further sodium alginate, propylene glycol alginate ester, CMC, sodium polyacrylate, methyl cellulose, soybean polysaccharides, and the like. The content of the optional thickening polysaccharide in the iron-containing composition of the present invention can be from 0.01 to 10 parts by mass, based on 100 parts by mass of the ferric pyrophosphate.

In the embodiment where an iron-containing composition of the present invention is in a liquid state, the content of water is, but not particularly limited to, preferably from 100 to 500 parts by mass, and more preferably from 150 to 400 parts by mass, based on 100 parts by mass of the ferric pyrophosphate.

The iron-containing composition of the present invention can contain other optional components, including emulsifying agents such as organic and fatty acid esters, such as monoglycerol fatty acid esters, diglycerol fatty acid esters, polyglycerol fatty acid esters, propylene glycol fatty acid esters, polyglycerol polyricinoleic acid esters, citric acid fatty acid esters, and succinic acid fatty acid esters, and lecithins such as enzymatically decomposed lecithins, enzymatically treated lecithins, and high-purity lecithins, excipients such as dextrins and crystalline celluloses, proteins such as casein, casein sodium, acid casein, micellar casein, soybean proteins, pea proteins, egg white powders, collagens, and gelatins, peptides or amino acids degrading these proteins, fats and oils such as vegetable oils, animal fats, processed fats and oils, and the like can be contained.

In the embodiment where an iron-containing composition of the present invention is a liquid, the viscosity thereof is influenced by the content of the ferric pyrophosphate or the like, and the viscosity is preferably 30000 mPa·s or less, and more preferably 10000 mPa·s or less, from the viewpoint of making an iron-containing composition producible, and when used in foodstuff, it is preferable that the composition is diluted to a viscosity of from 100 to 2000 mPa·s or so, and the like. In a case where a viscosity of an iron-containing composition as used herein is less than 10000 mPa·s, an iron-containing composition is prepared and held at 25° C. for 2 hours, and its viscosity is then measured with a BL-type viscometer manufactured by TOKI SANGYO CO., LTD. at 60 rpm. In addition, in a case where a viscosity of an iron-containing composition is 10000 mPa·s or more, an iron-containing composition is held at 25° C. for 2 hours, and its viscosity is then measured with a BH-type viscometer manufactured by TOKI SANGYO CO., LTD. at 20 rpm.

The water activity of the iron-containing composition of the present invention is preferably 0.85 or less, more preferably 0.83 or less, and even more preferably 0.80 or less, from the viewpoint of inhibiting the proliferation of bacteria. Although a lower limit thereof can be, but not particularly limited to, 0.10 or more, 0.30 or more, 0.50 or more, 0.70 or more, and the water activity may be within the range of any of the combinations of the above. The water activity of the iron-containing composition as used herein is measured by PAWKIT manufactured by METER.

The particle size (granularity) in the iron-containing composition of the present invention is preferably 550 nm or less, and more preferably 540 nm or less, and a lower limit thereof can be, but not particularly limited to, 150 nm or more, and the viscosity may be within the range of any of the combinations of the above. The particle size as used herein is measured with a dynamic light scattering type particle size distribution measurement apparatus Nano-ZS, manufactured by Malvern.

The iron-containing composition of the present invention can be used in foodstuff, feeds, cosmetics, pharmaceutical compositions, and the like. The foodstuff include, for example, wheat powder processed foods typically such as breads and noodles; rice processed products such as rice porridge and takikomi rice; snacks and confectioneries such as biscuits, cakes, candies, chocolate, Japanese rice crackers, arare (small rice biscuits), tablet candies, and wagashi (Japanese sweets); soybean processed foods such as tofu and processed foods thereof, beverages such as refreshing beverages, fruit juice beverages, milk beverages, lactic acid bacteria beverages, carbonate beverages, and alcoholic beverages; milk manufactured products such as yogurts, cheeses, butters, ice creams, coffee whiteners, whipped creams, and cow milks; seasonings such as soy sauce, miso (soybean paste), salad dressings, sauces, gravy sauces, margarine, and mayonnaise; livestock meat processed foods such as hams, bacons, and sausages; fishery processed foods such as kamaboko (fish sausage), hanpen (soft puffy fish cake), chikuwa (Japanese fish paste cooked in a bamboo-like shape), and canned foods of fish; oral and enteral nutritious diets such as concentrated fluid diets, semi-digested nutritious diets, and elemental diets, and the like. The feeds includes, for example, feeds for pets, livestock, cultivated fish, and the like. The cosmetics include lotions, milky lotions, bathing agents, and cleaning agents such as cleansing agents, toothpastes, and the like. The pharmaceutical composition can be widely used as medicaments, quasi-drugs, and the like. For example, the pharmaceutical composition can be used for the treatment or prevention of any diseases in want of supplementing or maintaining an iron content. Specifically, the pharmaceutical composition can be suitably used in applications of the treatment or prevention of iron-deficient anemia or sports anemia. Here, the pharmaceutical composition can also be prepared by blending together with other ingredients or the like having the same action as the iron-containing composition of the present invention. The form of the formulation of the pharmaceutical composition is not particularly limited so long as the iron-containing composition of the present invention is contained. Specific examples include abstracts, powders, finely pulverized products, granules, pills, capsules, tablets [including raw tablets, dragees, orally fast disintegrating tablets, chewable tablets, effervescent tablets, troche, film-coated tablets, etc.], dry syrups, films, liquids [including suspensions, milk agents, syrups, lemonades, etc.], jelly agents, and candy-forming agents [candies (drops), gummies, nougats, etc.]. Here, the capsule agent includes, besides the hard capsules, soft capsules in which a solution with an iron-containing composition of the present invention dispersed is filled.

The iron-containing composition of the present invention can be produced in accordance with a known method for production. For example, an iron-containing composition can be produced by blending ferric pyrophosphate, a polyhydric alcohol, gum arabic, and gum ghatti, and treating with a wet pulverizer such as a beads-mill, a sand-mill, or CoBall Mill, an emulsifier-disperser such as STAR BURST, Nanomizer, Microfluidizer, or Homogenizer, or a ultrasonic disperser. The blending amounts of various components, and the like are as mentioned above. Here, as the ferric pyrophosphate used as the raw material, a commercially available one can be used, and it is preferable that a ferric pyrophosphate having a specified electrical conductivity is used. Here, the ferric pyrophosphate used as the raw materials may be pulverized during the production steps so as to have a desired particle size, or raw materials previously finely pulverized may be used. The electrical conductivity of the ferric pyrophosphate is preferably 0.05 mS/cm or more, more preferably 0.08 mS/cm or more, even more preferably 0.10 mS/cm or more, even more preferably 0.20 mS/cm or more, and even more preferably 0.30 mS/cm or more, and an upper limit thereof can be, but not particularly limited to, for example, 1.5 mS/cm or less, and the electrical conductivity may be within the range of any of the combinations of the above. The electrical conductivity of the ferric pyrophosphate used as the raw material as used herein is an electrical conductivity in a 1% aqueous ferric pyrophosphate solution, and the electrical conductivity is measured with an electrical conductivity measurement meter ES-12, manufactured by HORIBA, Ltd. In addition, as the ferric pyrophosphate used as the raw material, it is preferable to use ferric phosphate in which the proportion of phosphorus contained in the supernatant obtained when the ferric pyrophosphate is washed with water is preferably 10% or less, and more preferably 5% or less. The proportion of phosphorus is measured by drying the supernatant, and measuring a dried product according to fluorescent X-ray analysis.

### EXAMPLES

The present invention will be described specifically hereinbelow by means of Examples and Comparative Examples, without intending to limit the present invention thereto.

Preparation of Iron-Containing Compositions

### Examples 1 to 11, and Comparative Examples 1 to 6

Four-hundred grams of ferric pyrophosphate A (electrical conductivity in a 1% aqueous ferric pyrophosphate solution: 0.40 mS/cm, a proportion of phosphorus contained in the supernatant obtained when washed with water: 1.4%) was blended and dispersed with 500 g of glycerol and 1100 g of ion-exchanged water, and the dispersion was subjected to a finely pulverizing treatment, to even out the sizes of the average particles of the ferric pyrophosphate to a size as listed in Table 1 or 2, to give a 20% by mass ferric pyrophosphate composition. The composition obtained was mixed with a thickening polysaccharide solution and glycerol in proportions as listed in Table 1 or 2, and the mixture was dispersed and homogenized with a homogenizer, to give an iron-containing composition (12.5% by mass ferric pyrophosphate dispersion). The resulting iron-containing composition was analyzed, and a volume-average particle size, a viscosity, and particle sizes (immediately after the preparation and 4 days after the preparation) of the ferric pyrophosphate were measured. In addition, with regard to each of Examples, its water activity was measured to confirm that the water activity was at a level in which the proliferation of bacteria could be inhibited. The results are shown in Tables 1 and 2.

<Dispersibility (Evaluation for Precipitations)>

A ferric pyrophosphate dispersion of each of Examples and Comparative Examples after 4 days from the preparation was added to a commercially available low fat milk so as to have an iron concentration of 5 mg/100 ml. The mixture was treated with Homo-mixer at 8000 rpm for 2 minutes, and then heated at 80° C. for 30 minutes, and allowed to stand in a refrigerator for one week, to evaluate the amount of yellow to yellow-brown precipitations in accordance with the following criteria.

### (Evaluation Criteria)

Score 5: The precipitations cannot be confirmed at all;
Score 4: The precipitations cannot be hardly confirmed;
Score 3: Some but small amounts of precipitations can be confirmed;
Score 2: The precipitations can be confirmed; and
Score 1: The precipitations can be confirmed in large amounts.

TABLE 1
Table 1

| | | Ex. 1 | Ex. 2 | Ex. 3 | Ex. 4 | Ex. 5 | Ex. 6 | Ex. 7 | Ex. 8 | Ex. 9 | Ex. 10 | Ex. 11 |
|---|---|---|---|---|---|---|---|---|---|---|---|---|
| Composition, | Ferric pyrophosphate A | 100 | 100 | 100 | 100 | 100 | 100 | 100 | 100 | 100 | 100 | 100 |
| parts by | Glycerol | 400 | 400 | 400 | 400 | 400 | 400 | 400 | 320 | 440 | 400 | 400 |
| mass | Gum ghatti | 20 | 18 | 15 | 16 | 22 | 15 | 16 | 20 | 20 | 20 | 20 |
| | Gum arabic | 0.4 | 2.4 | 5.4 | 4.4 | 0.44 | 0.3 | 0.32 | 0.4 | 0.4 | 0.4 | 0.4 |
| | Water | 279.6 | 279.6 | 279.6 | 279.6 | 277.6 | 284.7 | 283.7 | 359.6 | 239.6 | 279.6 | 279.6 |
| Ratio | Gum arabic/gum ghatti | 0.02 | 0.13 | 0.36 | 0.28 | 0.02 | 0.02 | 0.02 | 0.02 | 0.02 | 0.02 | 0.02 |
| Analytical | Average particle size, | 0.15 | 0.15 | 0.15 | 0.15 | 0.15 | 0.15 | 0.15 | 0.15 | 0.15 | 0.13 | 0.20 |
| results | μm | | | | | | | | | | | |
| | Water activity (Aw) | 0.74 | 0.75 | 0.74 | 0.75 | 0.75 | 0.74 | 0.75 | 0.84 | 0.71 | 0.75 | 0.74 |
| | Viscosity, mPa · s | 1433 | 1156 | 460 | 610 | 1650 | 530 | 630 | 1184 | 1884 | 1788 | 1200 |
| | Particle size | 504.7 | 508.1 | 499.6 | 491.9 | 501.8 | 446.8 | 453.0 | 502.9 | 510.8 | 480.3 | 510.3 |
| | immediately after the | | | | | | | | | | | |
| | preparation, nm | | | | | | | | | | | |
| | Particle size four days | 510.5 | 511.1 | 527.4 | 537.0 | 512.6 | 473.1 | 475.5 | 508.3 | 511.0 | 496.7 | 517.8 |
| | after the preparation, | | | | | | | | | | | |
| | nm | | | | | | | | | | | |
| Analytical | Dispersibility, | 5 | 4 | 4 | 4 | 5 | 4 | 5 | 5 | 5 | 5 | 4 |
| results, milk | evaluation for | | | | | | | | | | | |
| | precipitations | | | | | | | | | | | |

TABLE 2
TABLE 2

| | | Comp. | Comp. | Comp. | Comp. | Comp. | Comp. |
|---|---|---|---|---|---|---|---|
| | | Ex. 1 | Ex. 2 | Ex. 3 | Ex. 4 | Ex. 5 | Ex. 6 |
| Composition, | Ferric pyrophosphate A | 100 | 100 | 100 | 100 | 100 | 100 |
| parts by | Glycerol | 400 | 400 | 400 | 400 | 400 | 400 |
| mass | Gum ghatti | 20.4 | 8 | 0 | 24 | 14 | 20 |
| | Gum arabic | 0 | 12.4 | 20.4 | 0.48 | 0.28 | 0.4 |
| | Water | 279.6 | 279.6 | 279.6 | 275.5 | 285.7 | 279.6 |
| Ratio | Gum arabic/gum ghatti | 0 | 1.55 | | 0.02 | 0.02 | 0.02 |
| Analytical | Average particle size, | 0.15 | 0.15 | 0.15 | 0.15 | 0.15 | 0.25 |
| results | μm | | | | | | |
| | Viscosity, mPa · s | 1970 | 400 | 880 | 2410 | 380 | 270 |
| | Particle size immediately | 469.4 | 427.4 | 553 | 506.4 | 413.4 | 590 |
| | after the preparation, nm | | | | | | |
| | Particle size four days after | 554.6 | 607.1 | 898.6 | 563.0 | 448.1 | 621 |
| | the preparation, nm | | | | | | |
| Analytical | Dispersibility, evaluation for | 3 | 2 | 1 | 3 | 2 | 2 |
| results, milk | precipitations | | | | | | |

It can be seen from Tables 1 and 2 that the iron-containing compositions of Examples 1 to 11 are all excellent in dispersibility, as compared to the iron-containing compositions of Comparative Examples 1 to 3 in which the mass ratio of the content of gum arabic to the content of gum ghatti is outside the range of from 0.01 to 0.5, Comparative Examples 4 and 5 in which a total amount of gum arabic and gum ghatti is outside the range of from 15 to 23 parts by mass, and Comparative Example 6 in which the volume-average particle size of the ferric pyrophosphate exceeds 0.20 μm.

Preparation of Iron-Containing Compositions

### Examples 12 to 14

The same procedures as in Example 1 were carried out except that a 8.0% by mass ferric pyrophosphate dispersion was provided as a composition listed in Table 3, to give an iron-containing composition. In Table 3, ferric pyrophosphate A had an electrical conductivity of 0.40 mS/cm in a 1% aqueous ferric pyrophosphate solution, and a proportion of phosphorus contained in the supernatant obtained when washed with water of 1.4%; ferric pyrophosphate B had an electrical conductivity of 0.10 mS/cm in a 1% aqueous ferric pyrophosphate solution, and a proportion of phosphorus contained in the supernatant obtained when washed with water of 65.1%; and ferric pyrophosphate C had an electrical conductivity of 0.08 mS/cm in a 1% aqueous ferric pyrophosphate solution, and a proportion of phosphorus contained in the supernatant obtained when washed with water of 95.3%. With respect to the resulting iron-containing compositions, the evaluation for precipitations was carried out in the same manner as in Examples 1 to 11. In addition, the resulting iron-containing compositions were analyzed, and a volume average particle size, a viscosity, and particle sizes (immediately after the preparation and four days after the preparation) of the ferric pyrophosphates were measured. In addition, the water activity was measured, and it was confirmed to be at a level in which the proliferation of bacteria can be inhibited. The results are shown in Table 3.

TABLE 3

| | | Ex. 12 | Ex. 13 | Ex. 14 |
|---|---|---|---|---|
| Composition, | Ferric pyrophosphate A | 100 | 0 | 0 |
| parts by | Ferric pyrophosphate B | 0 | 100 | 0 |
| mass | Ferric pyrophosphate C | 0 | 0 | 100 |
| | Glycerol | 625 | 625 | 625 |
| | Gum ghatti | 20 | 20 | 20 |
| | Gum arabic | 0.4 | 0.4 | 0.4 |
| | Water | 322.9 | 322.9 | 322.9 |
| Ratio | Gum arabic/gum ghatti | 0.02 | 0.02 | 0.02 |
| Analytical | Average particle size, μm | 0.15 | 0.15 | 0.15 |
| results | Water activity (Aw) | 0.74 | 0.74 | 0.74 |
| | Viscosity, mPa · s | 128 | 1140 | 510 |
| | Particle size immediately | 503.9 | 508.2 | 516.9 |
| | after the preparation, nm | | | |
| | Particle size four days | 514.8 | 520.4 | 543.1 |
| | after the preparation, nm | | | |
| Analytical | Dispersibility, evaluation | 5 | 5 | 4 |
| results, milk | for precipitations | | | |

From Table 3, the dispersibility in all of the iron-containing compositions of Examples 12 to 14 was excellent, and the dispersibility of Examples 12 and 13, in which ferric pyrophosphates A, B, the ferric pyrophosphates having higher electrical conductivity were used was more excellent. In addition, when the viscosities were considered, the iron-containing composition of Example 12 blended with ferric pyrophosphate A was most excellent.

Preparation of Iron-Containing Compositions

### Examples 15 and 16

Five-hundred grams of ferric pyrophosphate A (electrical conductivity in a 1% aqueous ferric pyrophosphate solution: 0.40 mS/cm, a proportion of phosphorus contained in the supernatant obtained when washed with water: 1.4%) was blended and dispersed with 500 g of glycerol, 70 g of gum ghatti, 2 g of gum arabic, and 928 g of ion-exchanged water, and the dispersion was subjected to a finely pulverizing treatment for 60 minutes, to even out the sizes of the average particles of the ferric pyrophosphate to a size as listed in Table 4, to give an iron-containing composition of a 25% by mass ferric pyrophosphate. The resulting iron-containing composition was analyzed, and a volume-average particle size and a viscosity of the ferric pyrophosphate were measured. The results are shown in Table 4.

### Comparative Example 7

The same procedures as in Examples 15 and 16 were carried out except that the amount of glycerol and the amount of ion-exchanged water were changed to amounts as listed in Table 4, to try to prepare a composition. However, Comparative Example 7 was solidified so that a volume-average particle size and a viscosity could not be measured.

<Production Suitability>

The production suitability of Examples 15 and 16 and Comparative Example 7 was evaluated in accordance with the following criteria. The results are shown in Table 4.

### (Evaluation Criteria)

◯: producible as a liquid slurry during the finely pulverizing treatment;
Δ: thickening during the finely pulverizing treatment, but being producible as a liquid slurry; and
x: solidifying during the finely pulverizing treatment, making it non-producible, or having a volume-average particle size after the finely pulverizing treatment of exceeding 0.20.

TABLE 4

| | | | | Comp. |
|---|---|---|---|---|
| | | Ex. 15 | Ex. 16 | Ex. 7 |
| Composition, | Ferric pyrophosphate A | 100 | 100 | 100 |
| parts by | Glycerol | 100 | 100 | 90 |
| mass | Gum ghatti | 15 | 15 | 15 |
| | Gum arabic | 0.4 | 0.4 | 0.4 |
| | Water | 184.6 | 184.6 | 195.6 |
| Ratio | Gum arabic/gum ghatti | 0.03 | 0.03 | 0.03 |
| Analytical | Average particle size, μm | 0.15 | 0.20 | — |
| results | Viscosity, mPa · s | 4500 | 3800 | — |
| | Production suitability | ◯ | ◯ | X |

### Examples 17 and 18

To 100 parts by mass of an iron-containing composition of a 25% by mass ferric pyrophosphate obtained in Examples 15 and 16 were added 70 parts by mass of glycerol and 30 parts by mass of water to dilute, to prepare an iron-containing composition of a 12.5% by mass of ferric pyrophosphate. The resulting iron-containing compositions were subjected to the evaluation for precipitations in the same manner as in Examples 1 to 11. As a result, the precipitations were not visually seen in both the cases, confirming to show excellent dispersibility.

### Example 19

Four-hundred grams of ferric pyrophosphate A (electrical conductivity in a 1% aqueous ferric pyrophosphate solution: 0.40 mS/cm, a proportion of phosphorus contained in the supernatant obtained when washed with water: 1.4%) was blended and dispersed with 400 g of glycerol, 64 g of gum ghatti, 1.28 g of gum arabic, and 1134.72 g of ion-exchanged water, and the dispersion was subjected to a finely pulverizing treatment, to even out the sizes of the average particles of the ferric pyrophosphate to 0.15 μm, to give an iron-containing composition of a 20% by mass ferric pyrophosphate. To the solution were further added 80000 g of ion-exchanged water and 19134.72 g of dextrin, the mixture was heated to dissolve the dextrin, and the solution was spray-dried with a spray-dryer, to prepare a powdery iron-containing composition. The resulting iron-containing composition was subjected to the evaluation for precipitations in the same manner as in Examples 1 to 11. As a result, the precipitations were not visually seen, confirming to show excellent dispersibility.

Preparation of Iron-Containing Compositions

### Examples 20 to 23 and Comparative Examples 8 to 11

The same procedures as in Examples 15 and 16 were carried out except that the amount of each of the components was changed to the amount as listed in Table 5, to give each of iron-containing compositions of a 25% by mass ferric pyrophosphate. The resulting iron-containing compositions were analyzed, and a volume-average particle size and a viscosity of the ferric pyrophosphate were measured. The production suitability was evaluated in accordance with the same criteria as in Examples 15 and 16. The results are shown in Table 5. Here, Comparative Example 8 was solidified so that the volume-average particle size and the viscosity could not be measured.

TABLE 5
TABLE 5

| | | | | | | Comp. | Comp. | Comp. | Comp. |
|---|---|---|---|---|---|---|---|---|---|
| | | Ex. 20 | Ex. 21 | Ex. 22 | Ex. 23 | Ex. 8 | Ex. 9 | Ex. 10 | Ex. 11 |
| | Ferric pyrophosphate A | 100 | 100 | 100 | 100 | 100 | 100 | 100 | 100 |
| Composition, | Glycerol | 100 | 100 | 100 | 94 | 100 | 100 | 100 | 100 |
| parts by mass | Gum ghatti | 22.4 | 20 | 15.2 | 22.4 | 0 | 22.4 | 14.8 | 22.64 |
| | Gum arabic | 0.4 | 0.4 | 7.6 | 0.4 | 23 | 1.6 | 8 | 0.16 |
| | Water | 177.2 | 179.6 | 177.2 | 183.2 | 177 | 176 | 177.2 | 177.2 |
| Ratio | Gum arabic/gum ghatti | 0.02 | 0.02 | 0.50 | 0.02 | — | 0.07 | 0.54 | 0,007 |
| Analytical | Average particle size, μm | 0.15 | 0.15 | 0.15 | 0.15 | — | 0.3 | 0.25 | 0.3 |
| results | Viscosity, mPa · s | 29800 | 26000 | 24600 | 28600 | — | 31000 | 24000 | 34000 |
| | Production suitability | Δ | Δ | Δ | Δ | X | X | X | X |

As shown in Table 5, it could be confirmed that Examples 20 to 23 had production suitability at high concentrations even though the viscosities thereof would be near 30000, thereby making them less advantageous in the production efficiencies than Examples 15 and 16. On the other hand, although the same finely pulverizing treatment as in Examples 20 to 23 was carried out in Comparative Examples 9 to 11, particle aggregation was confirmed after the finely pulverizing treatment, thereby making them disadvantageous in dispersibility, so that they were evaluated as not having production suitability at high concentrations.

### Examples 24 to 27

To 100 parts by mass of an iron-containing composition of a 25% by mass ferric pyrophosphate each obtained in Examples 20 to 23 were added 70 parts by mass of glycerol and 30 parts by mass of water to dilute, to prepare each of iron-containing compositions of a 12.5% by mass ferric pyrophosphate. The resulting iron-containing compositions were subjected to the evaluation for precipitations in the same manner as in Examples 1 to 11. As a result, the precipitations were not visually seen in all the cases, confirming to show excellent dispersibility.

### INDUSTRIAL APPLICABILITY

The iron-containing composition of the present invention can be used in foodstuff, feeds, cosmetics, pharmaceutical compositions, and the like.

1-8. (canceled)

9. An iron-containing composition comprising ferric pyrophosphate, a polyhydric alcohol, gum arabic, and gum ghatti, wherein the content of the polyhydric alcohol is from 93 to 630 parts by mass, based on 100 parts by mass of the ferric pyrophosphate, and wherein a total amount of gum arabic and gum ghatti is from 15 to 23 parts by mass, based on 100 parts by mass of the ferric pyrophosphate, and wherein a mass ratio of the content of the gum arabic to the content of the gum ghatti (gum arabic/gum ghatti) is from 0.01 to 0.5, and wherein the ferric pyrophosphate has a volume-average particle size of 0.20 μm or less, and wherein the polyhydric alcohol comprises one or more members selected from the group consisting of propylene glycol, glycerol, diglycerol, triglycerol, and polyglycerol.

10. A method for producing an iron-containing composition comprising blending ferric pyrophosphate, a polyhydric alcohol, gum arabic, and gum ghatti, wherein the blending amount of the polyhydric alcohol is from 93 to 630 parts by mass, based on 100 parts by mass of the ferric pyrophosphate, and wherein a total amount of gum arabic and gum ghatti is from 15 to 23 parts by mass, based on 100 parts by mass of the ferric pyrophosphate, and wherein a mass ratio of the content of the gum arabic to the content of the gum ghatti (gum arabic/gum ghatti) is from 0.01 to 0.5, and wherein the ferric pyrophosphate has a volume-average particle size of 0.20 μm or less, and wherein the polyhydric alcohol comprises one or more members selected from the group consisting of propylene glycol, glycerol, diglycerol, triglycerol, and polyglycerol.

11. The method for production according to claim 10, further comprising a step of pulverizing ferric pyrophosphate.

12. The method for production according to claim 10, wherein ferric pyrophosphate having an electric conductivity of 0.05 mS/cm or more in a 1% aqueous ferric pyrophosphate solution is blended.

13. The method for production according to claim 10, wherein the blending amount of ferric pyrophosphate is from 15 to 25% by mass, and wherein the content of the polyhydric alcohol is from 93 to 330 parts by mass, based on 100 parts by mass of the ferric pyrophosphate.

14. Foodstuff comprising an iron-containing composition as defined in claim 9.
